(19) United States
(12) Patent Application Publication
(10) Pub. No.: US 2005/0271649 A1
(43) Pub. Date: Dec. 8, 2005

(54) Therapeutic adjuvant

(76) Inventors: Birgit C. Schultes, Arlington, MA (US); Christopher F. Nicodemus, Charlestown, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/531,849

(22) PCT Filed: Oct. 17, 2003
(86) PCT No.: PCT/US03/33027

§ 371 Date: Jul. 15, 2005

Related U.S. Application Data
(60) Provisional application No. 60/419,332, filed on Oct. 17, 2002.

(51) Int. Cl. A61K 39/395

(52) U.S. Cl. 424/130.1

(57) ABSTRACT

Disclosed are methods for enhancing an immune response associated with a disease or condition in a patient comprising administering a first composition comprising a non-specific xenotypic antibody and optionally further administering a second composition comprising a specific xenotypic antibody that specifically binds to an antigen associated with the disease or condition.

### RELATED APPLICATIONS

This application claims the benefit of priority of U.S. Provisional application 60/419,332, filed Oct. 17, 2002, which is incorporated herein in its entirety.

### BACKGROUND OF THE INVENTION

Diseases are often associated with overproduction of a specific cell-surface or circulating antigen. To date, antibody-mediated therapy typically consists of administering compositions of antibodies that specifically bind to the specific antigen causing the disease or condition.

Often, adjuvants are used to enhance the immune response to the antibody. They do so by increasing non-specific immune responses (e.g., inflammation), increasing the surface area of the antigen, prolonging the retention of the antigen in the body, retarding the release of antigen, targeting antigen to antigen presenting cells (e.g., macrophages and dendritic cells), activating antigen presenting cells, or otherwise eliciting non-specific activation of the cells of the immune system (Warren et al. Annu. Rev. Immunol. 4:369-388 (1986)).

Adjuvants utilizing non-specific mechanisms to enhance therapies have presented serious toxicities, interfered with subsequent therapies and/or diagnostics, or failed to demonstrate a meaningful therapeutic benefit.

### SUMMARY OF THE INVENTION

The invention provides a non-toxic adjuvant that is useful for producing or enhancing an effective immune response.

One embodiment of the present invention enhances an effective immune response in a patient suffering from a disease or condition, by administering to a patient a composition comprising a non-specific xenotypic antibody, wherein the non-specific xenotypic antibody does not specifically bind to an antigen associated with the disease or condition, and whereby an effective immune response is enhanced. The non-specific xenotypic antibody can be administered alone after the patient has already received administration of an antibody composition, specific antibody or non-specific antibody, or when the patient already has antibodies to the antigen associated with the disease or condition. In such a case, the non-specific xenotypic antibody would act as an adjuvant to enhance the specific immune response to the disease-associated antigen.

The method may further include administering to the patient a second composition comprising a second, preferably xenotypic, antibody that specifically binds to the disease-associated antigen (“specific xenotypic antibody”). The two compositions can be administered at the same time, or at different times. For example, the non-specific xenotypic antibody composition can be administered to the patient at a time prior to the specific xenotypic antibody composition (e.g., one day, one week, one month, etc.). Alternatively, the specific xenotypic antibody composition can be administered to the patient at a time prior to the non-specific xenotypic antibody composition (e.g., one day, one week, one month, etc.). If the compositions are administered at the same time, they can be formulated as a single composition or preferably, separate compositions.

In certain embodiments, the first non-specific xenotypic antibody enhances the effective immune response generated by the specific xenotypic antibody. The non-specific and specific xenotypic antibodies can be from the same or different species of animal as long as they are both from species different from the patient to be treated.

Xenotypic antibodies of the present invention can be monoclonal or polyclonal antibodies, humanized antibodies, chimeric antibodies, or fragments thereof, which are capable of producing or enhancing an effective immune response in a patient suffering from a disease or condition. Enhancement or production of an effective immune response can include: (1) generation of a host anti-xenotypic antibody (HAXA) response in the patient; (2) generation of a host anti-murine antibody (HAMA) response; (3) increased presentation of an antigen associated with the disease or condition by an antigen-presenting cell; (4) enhancement of an antigen-specific immune response, such as generation of a T cell that specifically recognizes the antigen (e.g., a CD4+ T cell response or a CD8+ T cell); and/or (5) enhancement of a humoral (e.g., B cell) response.

One embodiment of the present invention is a vaccine including a disease-associated antigen in combination with a non-specific xenotypic antibody of the present invention that non-specifically enhance the immune response produced in response to administration of the disease-associated antigen.

In certain embodiments, the pharmaceutical compositions of the present invention can be packaged as kits or pharmaceutical packages including instructions for use and labeled for use for the treatment of a patient having a disease or condition associated with an antigen.

Compositions of the present invention may further encompass a pharmaceutically acceptable carrier.

Composition of the present invention can be administered in a dosage of from about 0.1 μg to about 2 mg of the xenotypic antibody per kilogram of body weight of the patient.

### BRIEF DESCRIPTION OF THE DRAWINGS

FIG. 1 is a bar graph of the effect of complexation of antibody with specific antigen or human anti-mouse antibody (HAMA) on FITC-labeled antibody binding to dendritic cells. Both FITC-labeled Alt-2 and FITC-labeled Alt-6, two of the FITC-labeled murine monoclonal antibodies of the invention, exhibit enhanced binding in the presence of HAMA.

FIGS. 2A and 2B are bar graphs depict the binding of CA125-Alt-2 immune complexes by dendritic cells with or without HAMA. FIG. 2A depicts binding of FITC-labeled CA125 in the presence of 0, 0.313, 0.625, 1.25, and 2.5 μg/ml of Alt-2. FIG. 2B depicts binding of Alt-2-CA125 complexes to dendritic cells in the presence or absence of 0, 0.33, 1, or 2 μg/ml HAMA.

FIGS. 3A and 3B are bar graphs depicting the binding of CA125-Alt-2 immune complexes by monocytes in the presence of 0, 0.33, 1, and 2 μg/ml HAMA as measured by the percentage of positive events (FIG. 3A) and mean channel intensity (FIG. 3B). The uptake in FIGS. 3A and 3B was assessed for FITC-labeled CA125/Alt-2 complexes (light gray bars) or CA125/FITC-labeled Alt-2 complexes (dark gray bars in FIG. 3A; striped bars in FIG. 3B).

FIGS. 4A and 4B are bar graphs showing the uptake of CA125-Alt-2 immune complexes in monocytes (left two gray bars) and immature dendritic cells (right two white and black bars) in the presence (second and fourth bars from the left) and absence (first and third bars from the left) of HAMA as measured by the percentage of positive events (FIG. 4A) and mean channel intensity (FIG. 4B) for monocytes (left two gray bars) or immature dendritic cells (right two white and black bars).

FIG. 5 is a bar graph showing tumor volumes from three groups of SCID/bg mice with a reconstituted human immune system, carrying subcutaneous NIH:OVCAR-NU-3 tumors. Test antibodies or control agents were administered 7 days before and 7, 21 and 36 days following tumor cell implantation and included the following groups: 100 μg of Alt-2 (dotted bars), 100 μg of control antibody MOPC-21 (white bars) or PBS (black bars). Tumor measurements were taken during the course of the study in three dimensions and the tumor volumes calculated according to the formula: volume=π/6 Length*Width*Depth. FIG. 6 is a Kaplan-Meier survival plot showing survival times of three groups of SCID/bg mice with a reconstituted human immune system, carrying intraperitoneal NIH:OVCAR-NU-3 tumors. Test antibodies or control agents were administered 21 and 14 days prior to tumor cell implantation and 5, 19 and 34 days post tumor cell implantation and included the following groups: 100 μg of Alt-2 (bold dashed line), 100 μg of control antibody MOPC-21 (dotted line) or PBS (solid line). Animals were observed every other day for signs of distress, discomfort and presence of intra-abdominal tumor. Sick animals were euthanized and dissected to determine the presence and extend of tumor mass. Survival times are plotted by treatment group.

FIG. 7 is a bar graph showing tumor volumes from four groups of SCID/bg mice carrying subcutaneous NIH:OVCAR-NU-3 tumors. A human immune system was reconstituted in two groups two weeks after tumor implantation (first two bars). Test antibodies or control agents were administered on day 15 and 28 and included the following groups: 100 μg of MAb-B43.13 in mice with (white bars) and without (dotted bars) hPBL reconstitution, or 100 μg of MOPC 21 control antibody in mice with (hatched bars) and without (black bars) hPBL reconstitution by IP injections. Tumor volumes (mm³) were determined by three-dimensional tumor measurements every other day with a caliper in the various groups and tumor volumes calculated according to the formula: volume=π/6 Length*Width*Depth.

FIG. 8 is a Kaplan-Meier plot showing tumor appearance times in four groups of SCID/bg mice carrying subcutaneous NIH:OVCAR-NU-3 tumors. A human immune system was reconstituted in three of the four groups. Reconstituted mice were treated with 100 μg of Alt-2 (bold solid line) or 100 μg of MAb-170 control antibody (bold dashed line) or PBS (fine solid line). The fourth group of mice without hPBL reconstitution received no further treatment (fine dotted line). One week after reconstitution and antibody treatment, all mice were implanted with 2×10⁶ NIH:OVCAR-3 tumor cells by SC injection. The mice in groups 1 to 3 received a booster with respective antibodies at Days 4, 26 and 45. The time from tumor implantation to tumor appearance is plotted for each group.

FIG. 9 is a bar graph showing tumor weights from four groups of SCID/bg mice with a reconstituted human immune system, carrying subcutaneous NIH:OVCAR-NU-3 tumors. Test antibodies or control agents were administered 14 and 7 days before and 7, 14, and 21 days following tumor cell implantation and included the following groups: 50 μg of murine Alt-2, 50 μg of chimeric Alt-2, 50 μg of control antibody MOPC-21 or PBS. Tumors were excised at the end of the study and weighed. The mean tumor weight in each group is plotted.

FIG. 10 is a bar graph showing tumor weights from four groups of female BALB/c mice carrying subcutaneous MUC-1 expressing 413BCR tumors. Test antibodies or control agents were administered 14 and 7 days before and 7, 14, and 21 days following tumor cell implantation and included the following groups: 50 μg of murine Alt-1, human-chimeric Alt-1, non-specific mouse IgG control antibody (MOPC-21), non-specific chimeric control antibody and PBS. Tumors were excised at the end of the study and weighed. The mean tumor weight in each group is plotted.

### DETAILED DISCLOSURE OF THE INVENTION

### I. OVERVIEW

The present invention stems from the discovery that the presence of a host anti-xenotypic antibody response in a patient will enhance an immune response. Non-specific xenotypic antibodies have been developed for use as adjuvants, and are useful for enhancing or producing an effective immune response when administered alone, or in combination with an antibody specific for the antigen associated with a disease or condition.

Administration of the non-specific xenotypic antibodies alone is preferably used where a patient having a disease or condition associated with a particular antigen will receive, or has already received, treatment with an antibody specific for the disease-associated antigen (e.g., prostate specific antigen), or already has developed antibodies specific for the disease associated antigen. Thus, the non-specific xenotypic antibodies do not specifically bind to the disease-associated antigen and are referred to as non-specific.

### II. EXEMPLARY DEFINITIONS

As used herein, the term “disease associated with an antigen” means a disease or condition in which signs or symptoms of illness in a majority of patients are present when the antigen (e.g., prostate-specific antigen) is present in the patient's body at a certain concentration, but in which signs or symptoms of illness are absent or reduced when the antigen is absent from the patient's body or present in the patient's body at a lower concentration. “Signs of illness” or “symptoms of illness” are clinically recognized manifestations or indications of a disease state.

As used herein, the terms “antigen associated with a disease” and “disease-associated antigen” mean an antigen with which the disease state is associated, such as prostate-specific antigen.

The terms “species” and “animal” refers to mammals, preferably mammals such as humans. Likewise, a “patient” or “subject” to be treated by the method of the invention can mean either a human or non-human animal. In certain embodiments, the patient is a human. In other embodiments, the patient is preferably a non-human mammal, particularly a laboratory animal. Preferred non-human patients of the invention include, without limitation, mice, rats, rabbits, non-human primates (e.g., chimpanzees, baboons, rhesus monkeys), dogs, cats, pigs, horses, cows, and armadillos.

“Immune complex” or “immunogenic complex” as used herein mean an antibody/antigen complex.

As used herein, a “xenotypic antibody” is an antibody from a species other than the patient's species. For example, if the patient is a mouse, a rat antibody is a xenotypic antibody. The term “antibody” encompasses any molecule that includes a portion encoded by a immunoglobulin gene, or fragment thereof (e.g., Fc, Fab, Fv, and F(ab′)₂), and such antibody derived molecules comprise at least one variable region (either a heavy chain of or a light chain variable region), as well as individual antibody light chains, individual antibody heavy chains, chimeric fusions between antibody chains and other molecules, and the like. Also encompassed by the term “antibody” are polyclonal antibodies, monoclonal antibodies (“MAb”), preferably IgG1 antibodies; chimeric monoclonal antibodies (“C-MAb”); humanized antibodies (“H-Ab”); genetically engineered monoclonal antibodies (“G-MAb”).

“An active portion of an antibody” is a molecule that includes a binding site that is specific for an antigen. Alternatively, an “active portion of an antibody” is a molecule that includes a receptor binding site that binds a receptor on dendritic cells with its ligand-binding site (e.g., the Fc portion of the antibody including the heavy chain constant region or the carbohydrate chain at the hinge region). Accordingly, an antibody fragment of the invention may be, e.g., chimeric, single chain, or mutated. Fragments of antibodies can be non-covalently or covalently bonded to another moiety. For example, a Fab fragment can be joined to a tag or chemical (e.g., biotin). Alternatively, the Fc portion of an antibody joined to a peptide tag or a chemical.

As used herein, “administering” means providing the composition to the patient in a manner that results in the composition being inside the patient's body. Such an administration can be by any route including, without limitation, subcutaneous, intradermal, intravenous, intra-arterial, intraperitoneal, and intramuscular routes of administration. Compositions of the present invention can be administered conjointly (e.g., in the same formulation, or in different formulations administered at the same time) or administered separately.

A physician or veterinarian having ordinary skill in the art can readily determine and prescribe the “effective amount” (ED₅₀) of the pharmaceutical composition required. For example, the physician or veterinarian could begin by administering doses of the compounds of the invention employed in the pharmaceutical composition at levels lower than that required in order to achieve the desired therapeutic effect and gradually increase the dosage until the desired effect is achieved.

The phrase “therapeutically effective amount” as used herein means that amount of a compound, material, or composition comprising a compound of the present invention which is effective for producing some desired therapeutic effect by increasing or enhancing immune responses in a patient, at a reasonable benefit/risk ratio applicable to any medical treatment.

An “effective immune response” is defined herein wherein the patient experiences partial or total alleviation or reduction of signs or symptoms of illness, and specifically includes, without limitation, prolongation of survival. The patient's symptoms may remain static, and the disease symptoms may not increase. Further, an effective immune response is the production and/or enhancement of an effective B and/or T cell response. The T cell response may be a T helper response, a CTL response, or both a T helper and a CTL response. “Induction of a T helper response” is defined herein as causing T helper cells to provide the support to B cells or CTL such that an effective antibody or cytolytic response is induced and/or enhanced.

Each of the embodiments of the present invention can be used as a composition when combined with a pharmaceutically acceptable carrier or excipient. “Carrier” and “excipient” are used interchangeably herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier” is defined herein as a carrier that is physiologically acceptable to the administered patient and that retains the therapeutic properties of the antibody with which it is administered. Pharmaceutically acceptable carriers and their formulations are well known and generally described in, for example, Remington's Pharmaceutical Sciences (18^{th} Edition, ed. A. Gennaro, Mack Publishing Co., Easton, Pa., 1990). On exemplary pharmaceutically acceptable carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject binding agents or treated dendritic cells from the administration site of one organ, or portion of the body, to another organ, or portion of the body. Each carrier must be “acceptable” in the sense of being compatible with the other ingredients of the formulation and not injurious to the patient. Nor should a pharmaceutically acceptable carrier alter the specific activity of the binding agents of treated dendritic cells. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A “therapeutic benefit” means that the patient administered the composition of the invention experiences alleviation or reduction of signs or symptoms of the disease and/or experiences alleviation or reduction of the antigen. The phrase “a therapeutic benefit” specifically includes, without limitation, prolongation of survival.

As used herein, “antigen-specific immune response” and “specific immune response” mean that the patient develops an immune response, hallmarked by T cells, B cells, or preferably both, against a particular antigen, such that the concentration of the antigen in the patient is reduced as compared to the concentration of the antigen in a patient who did not receive the composition. Preferably, the reduction is at least 2-fold. Preferably, a patient who has developed an antigen-specific immune response comprises at least a T cell that specifically recognizes the antigen and a B cell (or antibody) that specifically binds to the antigen.

As used herein, “immunoreactive” refers to binding agents, antibodies or fragments thereof that are specific to a target antigen, yet if they are cross-reactive to other proteins, are not toxic at the levels at which they are formulated for administration to human use. “Specifically binds” means that the binding agent binds to the antigen with greater affinity than it binds unrelated antigens. Preferably such affinity is at least 10-fold greater, more preferably at least 100-fold greater, and most preferably at least 1000-fold greater than the affinity of the binding agent for unrelated antigens. Preferably, a patient's antibody that specifically binds to an antigen forms an association with that antigen with an affinity of at least 10⁶ M⁻¹, more preferably, at least 10⁷ M⁻¹, even more preferably, at least 10⁸ M⁻¹, even more preferably, at least 10⁹ M⁻¹, and most preferably, at least 10¹⁰ M⁻¹ either in water, under physiological conditions, or under conditions which approximate physiological conditions with respect to ionic strength, e.g., 140 mM NaCl, or 5 mM MgCl₂. For example, a patient's antibody, either freely circulating in the blood or cell-bound (e.g., cell-surface immunoglobulin, or bound to an Fc receptor on a mast cell or APC), binds to the indicated antigen with greater affinity than it binds unrelated proteins. The terms “immunoreactive” and “specifically binds” are used interchangeably herein.

As used herein, “specifically recognizes” mean that a patient's T cell binds to and responds to an antigen or fragment thereof presented in context of an MHC complex molecule that is syngeneic to the patient. Thus, a T cell that specifically recognizes an antigen will bind to the fragment peptide and the MHC complex molecule presenting the antigen and respond by either proliferating, secreting cytokines, and/or changing its cell surface phenotype (e.g., up-regulating CD69 expression). Thus, a patient's T cell that specifically recognizes the antigen will bind to and respond to the antigen when the antigen is presented on a B cell isolated from the patient.

By “syngeneic” MHC molecule is meant that the MHC molecule is found on a cell in the patient. Accordingly, syngeneic MHC molecules of the invention include all of those found on a cell in a patient. Additional syngeneic MHC molecules of the invention include all of those found on a cell in a patient. Additional syngeneic MHC molecules of the invention include those found on cells of the patient's blood relative (e.g., siblings, parents, offspring). It will be understood that not all of the MHC molecules on, for example, a fraternal sibling of a patient will be syngeneic to the patient. Of course, all of the MHC molecules on a cell of an identical sibling are syngeneic to the patient. Likewise, two individuals of an inbred strain of laboratory animal (e.g., inbred BALB/c mice) can provide syngeneic MHC molecules to one another. The type of MHC expressed by the patient depends, of course, on the species of the patient (e.g., humans express HLA antigens while mice express MHC antigens). It will be understood that to be syngeneic, the MHC must be from the same species of animal as the patient.

As used herein “increased immune response” or “enhanced immune response” mean that a patient's immune response, specific or non-specific, is increased as compared to the immune response in the patient prior to the administration of the non-specific xenotypic antibody or to the immune response in a control patient not treated with the non-specific xenotypic antibody. Standard methods of detecting an increased immune response are known, such as an ELISA assay, a Radioimmunoassay, or by Western Blot. For example, the antibody titer of a patient may be determined before and after the patient is administered the non-specific xenotypic antibody according to the methods of the invention, where an increased number of white blood cells after administration as compared to before administration indicates an increased immune response. Administration of the non-specific xenotypic antibody that results in an increased immune response in the patient, which may be specific or non-specific, allows the patient's immune response to be specifically stimulated against an antigen, preferably an antigen associated with a disease.

As used herein, the term “cancer” is used to mean a condition in which a cell in a patient's body undergoes abnormal, uncontrolled proliferation. Non-limiting examples of cancers include leukemias, multiple myelomas, prostate, ovarian, testicular, breast, or lung tumor, melanomas, lymphomas, etc. As used herein, the term “cancer” refers to any neoplastic disorder, including such cellular disorders as, for example, renal cell cancer, Kaposi's sarcoma, chronic leukemia, breast cancer, sarcoma, ovarian carcinoma, rectal cancer, throat cancer, melanoma, colon cancer, bladder cancer, mastocytoma, lung cancer, mammary adenocarcinoma, pharyngeal squamous cell carcinoma, gastrointestinal or stomach cancer, epithelial cancer, or pancreatic cancer.

As used herein, “transformed cells” refers to cells that have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. For purposes of this invention, the terms “transformed phenotype of malignant mammalian cells” and “transformed phenotype” are intended to encompass, but not be limited to, any of the following phenotypic traits associated with cellular transformation of mammalian cells: immortalization, morphological or growth transformation, and tumorigenicity, as detected by prolonged growth in cell culture, growth in semi-solid media, or tumorigenic growth in immuno-incompetent or syngeneic animals.

The term “sample” is defined herein as blood, blood product, biopsy tissue, serum, or any other type of fluid or tissue that can be extracted from a patient.

“Host anti-xenotypic antibodies (HAXA)” are antibodies of the host animal species that bind to the xenotypic antibody contained in the composition of the invention. For example, if the patient is a human and the xenotypic antibodies are rabbit antibodies, then the HAXAs are human anti-rabbit antibodies. Preferred HAXAs include, without limitation, human anti-mouse antibodies (HAMA).

The term “antigen-presenting cell” means any cell that is capable of presenting an antigen in the context of both MHC class I and MHC class II. Accordingly, an antigen-presenting cell (APC) is capable of presenting an antigen to both CD4⁺ T cells (which are mostly helper T cells) and CD8⁺ T cells (which are mostly cytotoxic T cells). Antigen-presenting cells of the invention thus include, without limitation, B cells, macrophages, monocytes, and dendritic cells (DCs). A “dendritic cell” is defined herein as a bone marrow-derived cell that can internalize antigen and process the antigen such that it (or a peptide derived from an antigen of the tumor cell) is presented in the context of both the MHC class I complex and the MHC class II complex. It should be understood that any cell capable of presenting a peptide derived from an internalized antigen on both class I and class II MHC is a dendritic cell of the invention. Preferably, a dendritic ell of the invention has the phenotype and characteristics of the dendritic cells described in Steinman, Annu. Rev. Immunol. 9: 271-296 (1991). “Immature dendritic cells” are defined herein as a population of dendritic cells having preferably one or more of the cell surface antigens at the indicated level of expression as described in PCT application WO 01/85204 by Schultes et al. “Precursor dendritic cells” are defined herein as a population of cells, each of which is capable of becoming a dendritic cell, e.g., monocytes, where greater than 80% of the population have CD64 and CD32 antigen present and about 70% of the population is positive for CD14.

The term “increases presentation of the antigen” means that a patient's antigen presenting cell has an increased ability to present antigen in context of class I and/or class II MHC after the patient has been administered a composition of the invention as compared to the ability of the cell to present antigen before the patient was administered the composition of the invention.

A “disease-associated antigen” is an antigen in the patient's body secreted and/or produced by a cell, and which may be presented on a surface, or circulating in the patient's bloodstream, or both. Preferred disease-associated antigens are tumor-associated antigens, such as CA125, PSA, MUC-1, CA19.9, and TAG-72.

As used herein, a “pathogen” is an agent capable of causing disease. Preferred pathogens include, for example, viruses (e.g., hepatitis B, hepatitis C, herpes and HIV-1), viroids, bacteria (e.g., Escherichia species Streptococcus species, Staphylococcus species, Enterococcus species), fungi (e.g., Amanita species, Agaricus species, Neolentinus species, Tricholoma species, etc.), prions, and parasites.

“After administration of the composition” means anytime after the administration of the composition comprising the non-specific xenotypic antibody. Preferably, analysis of the patient receiving administration of the compositions of present invention occurs within 3 years, 2 years, 1 year, 6 months, 3 months, or 1 month after administration of the composition.

By “pharmaceutically acceptable carrier” is meant a carrier that is physiologically acceptable to the administered patient and that retains the therapeutic properties of the antibody with which it is administered. One exemplary pharmaceutically acceptable carrier is physiological saline. Other pharmaceutically acceptable carriers and their formulations are well-known and generally described in, for example, Remington's Pharmaceutical Sciences (18^{th} Edition, ed. A. Gennaro, Mack Publishing Co., Easton, Pa., 1990).

### III. EXEMPLARY EMBODIMENTS

A. Compounds and Compositions

In one aspect, a composition comprises a xenotypic antibody that does not specifically bind to the disease-associated antigen (i.e., a non-specific xenogeneic antibody). In a further embodiment, the antibody is a xenotypic monoclonal antibody. In another embodiment, the composition is a fragment of a xenotypic antibody (e.g., Fc, Fab, scFv, Fv, and F(ab′)₂).

Preferred xenotypic antibodies of the invention include monoclonal or polyclonal antibodies, or a mixture of both. For example, a monoclonal murine antibody that specifically binds to a non-human protein (e.g., chicken egg albumin) can be used as a xenotypic antibody in a human patient. Similarly, a polyclonal antibody can be used as a xenotypic antibody in a human patient. For example, where the patient is a human the composition may include some polyclonal rabbit antibodies of various isotypes (e.g., IgM, IgA, and IgG), and some rabbit monoclonal antibodies of a single isotype (e.g., IgG). The complementary determining regions (CDRs) of the xenotypic antibodies of the present invention can be modified such that the antibody is humanized. Methods of humanizing antibodies are well known in the art.

Yet another xenotypic antibody of the invention is a chimeric antibody, where one of the portions of the chimeric antibody is xenotypic to the species to which the antibody is administered and the other portion is not. For example, where the patient is human, a chimeric antibody comprising a human Fc portion or human heavy or light chains.

In a further embodiment, the antibody or fragment can be linked to a second moiety, such as a tag, a chemical, a fluorescent label, etc. For example, a molecule consisting of the Fab portion of an antibody can be joined to a purification tag or chemical (e.g., biotin). Fragments of antibodies that contain the binding region can also be humanized using methods known to a person of ordinary skill in the art using routine methodology. Alternatively, a murine Fab antibody fragment can be joined by a covalent peptide bond to a synthetic peptide tag, such as a HA-Tag (e.g., amino acid sequence: YPYDVPDYA (SEQ ID NO.1)), a Myc-tag (e.g., amino acid sequence: EQKLISEEDL (SEQ ID NO.2), or a glutathione-S-transferase (GST) tag, using a pGEX plasmid (commercially available from Amersham Pharmacia Biotech, Inc., Piscataway, N.J.) using recombinant DNA technology. For example, a histidine tag (his-tag) can be attached to the Fab portion of Alt-2, thereby generating a nucleic acid molecule encoding the his-tagged polypeptide, and expressing the polypeptide in a cell (e.g., in COS or HeLa cells). This his-tagged polypeptide, once expressed, is readily purified by standard techniques (e.g., using the TALON™ Resin commercially available from Clontech Laboratories, Inc., Palo Alto, Calif.). Similarly, the Fc portion of an antibody can be joined to a peptide tag or a chemical as described above. Thus, a xenotypic antibody of the invention may or may not comprise a Fab portion. In one embodiment wherein the patient is a human, the Fc region of a murine antibody can be covalently bonded to a chemical, such as biotin, thereby allowing for purification of the murine Fc region on a streptavidin column.

Preferably, the non-specific and specific xenotypic antibodies in the composition of the invention are from one species; however, the xenotypic antibodies in the composition can be from several different species so long as each species is different from that of the patient. Accordingly, where the patient is human, the composition may comprise xenotypic antibodies that are all murine. Alternatively, when the patient is human, the composition may comprise xenotypic antibodies from several different non-human species, such as mice, rats, non-human primates, and rabbits. Preferred xenotypic antibodies of the invention are monoclonal antibodies. Where the patient is human, these xenotypic monoclonal antibodies include, without limitation, murine monoclonal antibodies. Particularly preferred murine monoclonal antibodies include Alt-1 (murine IgG1, specifically binds to MUC-1), Alt-2 (murine IgG1, specifically binds to CA125), Alt-3 (murine IgG3, specifically binds to CA19.9), Alt-4 (murine IgM, specifically binds to CA19.9), Alt-5 (murine IgG1, specifically binds to CA19.9); and Alt-6 (murine IgG1, specifically binds to prostate specific antigen (PSA)).

The mouse hybridoma AR20.5R8223, which makes the antibody Alt-1, was deposited with the American Type Culture Collection, 10801 University Blvd., Manassas, Va. 20110-2209, on, Nov. 23, 1999, and was given ATCC deposit number PTA-975.

The mouse hybridoma B43.13, which makes the antibody Alt-2, was deposited with the American Type Culture Collection, 10801 University Blvd., Manassas, Va. 20110-2209, on May 18, 2000, and was given ATCC deposit number PTA-1883.

The mouse hybridoma AR44.6R1331, which makes the antibody Alt-3, was deposited with the American Type Culture Collection, 10801 University Blvd., Manassas, Va. 20110-2209, on Nov. 17, 2000, and was given ATCC deposit number PTA-2691.

The mouse hybridoma AR18.4R3313, which makes the antibody Alt-4, was deposited with the American Type Culture Collection, 10801 University Blvd., Manassas, Va. 20110-2209, on Nov. 17, 2000, and was given ATCC deposit number PTA-2692.

The mouse hybridoma AR44.3R15, which makes the antibody Alt-5, was deposited with the American Type Culture Collection, 10801 University Blvd., Manassas, Va. 20110-2209, on Nov. 17, 2000, and was given ATCC deposit number PTA-2690.

The mouse hybridoma AR47.47R66, which makes the antibody Alt-6, was deposited with the American Type Culture Collection, 10801 University Blvd., Manassas, Va. 20110-2209, on Apr. 29, 1998, and was given ATCC deposit number HB-12526.

All deposited hybridomas are being maintained under the conditions of the Budapest Treaty and will be maintained for a period of at least 30 years from date of deposit, or five years after the most recent request for a sample, whichever is longer.

One aspect of the present invention includes compositions formulated in pharmaceutically acceptable carriers that can be administered to a patient. On exemplary pharmaceutically acceptable carrier is physiological saline. Other pharmaceutically-acceptable carriers and their formulations are well-known and generally described in, for example, Remington's Pharmaceutical Sciences (18^{th} Edition, ed. A. Gennaro, Mack Publishing Co., Easton, Pa., 1990). In a further embodiment, the pharmaceutical preparations (e.g., compositions) are substantially free from pyrogens.

Another aspect of the present invention is the use of the antibodies in the preparation of a medicament for the treatment of patients suffering from a disease wherein an effective immune response is elicited and/or enhanced.

B. Methods of Treatment

In one embodiment, the invention provides a method for enhancing an effective immune response in a patient suffering from a disease or condition, comprising administering to a patient a composition comprising a non-specific xenotypic antibody, whereby an effective immune response is enhanced. Thus, the xenotypic antibody is non-specific with respect to the disease or condition being treated and acts as an adjuvant to non-specifically enhance an effective immune response. One skilled in the art will recognize that patients receiving treatment and who have an ongoing therapeutic immune response would be candidates to receive administration of the non-specific xenotypic antibody to enhance the effective immune response in a single dose or multiple doses.

In certain embodiments, the patient produces or is administered an antibody that specifically binds to the disease-associated antigen (i.e., a specific antibody), the specific antibody could be the patient's own antibody, or an antibody that has been administered to the patient. For example, a patient suffering from HIV infection may already have generated his/her own antibodies that specifically bind to an antigen from the HIV virus (e.g., gp120). Similarly, a patient suffering from cancer may already have generated antibodies that specifically bind to a tumor-associated antigen. In these examples, administration of a non-specific xenotypic antibody augments the ability of the patient's own specific antibody to provide a therapeutic benefit to the patient.

The methods according to the invention are useful for therapeutically treating patients having a disease or condition associated with an antigen. Some non-limiting examples of disease-associated antigens include the prostate specific antigen (associated with prostate cancer), BRCA-1 and BRCA-2 antigens (associated with many adenocarcinomas, including breast cancer, lung cancer, and pancreatic cancer), CA125 (associated with ovarian cancer), aberrant myelin basic protein (associated with multiple sclerosis), gp120 (associated with colorectal, stomach, and pancreatic cancers), TAG-72 (associated with ovarian, stromal, and pancreatic cancers) and p53 (associated with various cancers).

Asymptomatic patients are those having a disease-associated antigen in the patient's body, yet failing to exhibit symptoms of the disease. For example, an elevated CA125 level (e.g., detected in the serum at a concentration greater than 35 units/ml) in a patient may not yet be symptomatic for ovarian cancer, yet would be a candidate for treatment with a non-specific xenotypic antibody of the present invention.

Non-specific xenotypic antibodies of the present invention can be administered by any route including, without limitation, subcutaneous, intradermal, intravenous, intra-arterial, intraperitoneal, and intramuscular routes of administration.

A patient who has been treated with a non-specific xenotypic antibody may experience alleviation or reduction of signs or symptoms of the disease and/or alleviation or reduction of the antigen level. Treatment with non-specific xenotypic antibodies of the present invention may also prolong survival of the patient.

Thus, in one exemplary embodiment of the invention, a human patient suffering from prostate cancer who has circulating prostate specific antigen (PSA) and anti-PSA antibodies is administered a non-specific xenotypic antibody (e.g., a murine monoclonal antibody or a rabbit polyclonal antibody). The non-specific xenotypic antibody acts as an adjuvant in producing an effective immune response by eliciting host anti-xenotypic antibodies (HAXA) in the patient. Additionally, antigen presentation by an antigen-presenting cell in the patient may increase, for example, because the xenotypic antibody facilitates uptake of the antigen by the patient's antigen-presenting cells.

In a further embodiment, the patient is administered a second composition comprising a specific xenotypic antibody that specifically binds to the disease-associated antigen. The specific xenotypic antibody may be either a monoclonal or a polyclonal antibody. For example, where the disease associated with an antigen is prostate cancer, a murine monoclonal antibody that specifically binds to prostate specific antigen (PSA) can be used as the specific xenotypic antibody of the invention. In another non-limiting example, goat polyclonal antibody raised against human PSA antigen can be used as the specific xenotypic antibody of the invention.

Specific antibodies of the present invention to be given in conjunction with the non-specific xenotypic antibody composition are those that are immunoreactive with a disease-associated antigen, and which bind to the indicated antigen with greater affinity than unrelated proteins. Preferably such affinity is at least 10-fold greater than the affinity of the binding agent for unrelated proteins. Preferably, a patient's antibody that specifically binds to an antigen forms an association with that antigen with an affinity of at least 10⁶ M⁻¹, more preferably, at least 10⁷ M⁻¹, even more preferably, at least 10⁸ M⁻¹, even more preferably, at least 10⁹ M⁻¹, and most preferably, at least 10¹⁰ M⁻¹ either in water, under physiological conditions, or under conditions which approximate physiological conditions with respect to ionic strength, e.g., 140 mM NaCl, or 5 MM MgCl₂. Specific antibodies can be freely circulating in the blood, or bound to a cell surface (e.g., cell-surface immunoglobulin on a B cell, such as an IgM or IgD, or bound to a Fc receptor on a cell, such as a mast cell or macrophage).

In certain preferred embodiments, the disease-associated antigen is from a pathogen capable of causing disease. Preferred pathogens include, for example, viruses (e.g., hepatitis B, hepatitis C, herpes and HIV-1), viroids, bacteria (e.g., Staphylococcal species, Enterococcal species, Streptococcal species, etc.), fungi, prions, and parasites.

In certain embodiments, antigen may be circulating throughout the patient.

In other embodiments, the antigen may be localized to the area of the body effected the disease (e.g., a tumor antigen associated with a benign tumor). In yet another embodiment, the antigen may be a cell-surface antigen. Thus, in certain preferred embodiments, the antigen is a tumor-associated antigen (i.e., an antigen in the patient's body secreted and/or produced by tumor cells, and which may be presented on the tumor surface, or circulating in the patient's bloodstream, or both). Preferred tumor-associated antigens include, without limitation, CA125, PSA, MUC-1, CA19.9, and TAG-72.

In one embodiment, the non-specific antibody is administered to the patient prior to administration of the composition comprising a specific xenotypic antibody. In such a scenario, the non-specific xenotypic antibody has elicited a host anti-xenotypic antibody response in the patient before the patient is administered the specific xenotypic antibody immunoreactive with the disease-associated antigen. Preferably, the specific xenotypic antibody is from the same species of animal as the non-specific xenotypic antibody. Thus, where the specific xenotypic antibody is administered to the patient subsequent to the non-specific xenotypic antibody, the non-specific xenotypic antibody of the first composition elicits a HAXA response in the patient prior to the administration of the second specific xenotypic antibody. For example, a female human patient suffering from breast cancer expressing MUC-1 is administered a murine antibody immunoreactive with prostate specific antigen (PSA) (i.e., a non-specific xenogeneic antibody). Once the female patient generates a HAMA response as readily determined using standard methods, the patient is administered a murine antibody that specifically binds to MUC-1 (i.e., a specific xenogeneic antibody).

Alternatively, in another embodiment, the specific antibody is administered to the patient prior to administration of the composition comprising a non-specific xenotypic antibody. Preferably, the specific xenotypic antibody of the first composition and the non-specific xenotypic antibody of the second composition are from the same species of animal.

In one embodiment, a patient suffering from a disease associated with the CA125 antigen (e.g., ovarian cancer) is administered an anti-CA125 antibody (i.e., an antibody that specifically binds to CA125). Subsequent administration of a non-specific xenotypic antibody of the invention augments the anti-CA125 antibody to provide a therapeutic benefit to the patient. For example, where two patients both receive the anti-CA125 antibody therapy, and one patient subsequently receives a non-specific xenotypic antibody of the invention, the patient who subsequently received a non-specific xenotypic antibody of the invention may have a better prognosis (e.g., remission, reduction in tumor size, or an enhanced effective immune response) as compared to the patient who received only the anti-CA125 antibody.

Similarly, in certain embodiments where the composition comprising a specific xenotypic antibody is administered prior to administration of the second composition comprising a non-specific xenotypic antibody, the specific xenotypic antibody of the composition elicits a HAXA response in the patient prior to the administration of the non-specific antibody. For example, a female human patient suffering from breast cancer and expressing MUC-1 is administered a murine antibody that specifically binds to MUC-1. Once the female patient generates a HAMA and an anti-MUC-1 response (as readily determined using standard methods), the patient is administered a murine antibody that specifically binds to PSA. Accordingly, after administration of the specific xenotypic antibody, the patient also generates an antigen-specific immune response comprising a T cell that specifically recognizes PSA, a B cell (and antibodies) that specifically bind to PSA or both, and induction of this immune response augments the response to MUC-1 by providing cytokines and signals to activate antigen-presenting cells.

In a preferred embodiment, where the composition comprising a non-specific xenotypic antibody is administered to the patient after the composition comprising a specific xenotypic antibody, the specific xenotypic antibody does not elicit a HAXA response in the patient prior to the administration of the non-specific xenotypic antibody. For example, a female human patient suffering from ovarian cancer and expressing CA125 is administered a murine antibody that specifically binds to CA125. However, the patient does not generate a HAMA response. The patient is then administered a murine antibody that specifically binds to PSA, the non-specific xenotypic antibody. The administration of the anti-PSA antibody may then elicit a HAMA response in the patient, e.g., an enhanced effective immune response. The patient may have a better prognosis following administration of the anti-PSA murine antibody (i.e., adjuvant) than administration of only the anti-CA125 murine antibody.

Additionally, the compositions may be administered at the same time as individual compositions, or may also be combined to be administered together as a single composition.

In certain embodiments, the non-specific and specific xenotypic antibody compositions are administered together. In one exemplary embodiment, a female human patient suffering from breast cancer expressing MUC-1 is administered a mixture of a xenotypic antibody that specifically binds to MUC-1 and a xenotypic antibody that specifically binds to PSA. The patient receiving both anti-MUC-1 and anti-PSA xenotypic antibodies may have a better prognosis than a patient who received only an anti-MUC-1 xenotypic antibody.

In certain embodiments, the invention provides a method for enhancing an effective immune response, which may be directed against, for example, a xenotypic antibody or a disease-associated antigen, comprising administering to the patient a low dose or a small amount of a non-specific xenotypic antibody. In some embodiments of the invention, the low dose of the non-specific xenotypic antibody comprises from about 0.1 μg to about 2 mg per kg of body weight of the patient to be treated. The presence of the generation of an enhanced immune response can be measured using standard techniques (e.g., increase in white blood cell count; increase in circulating antibody levels, increase in T cell responses). Where the immune response is a HAXA response, the presence of the HAXA can be determined, for example, by ELISA.

In some embodiments, about I1 μg/kg to about 1.0 mg/kg of the non-specific xenotypic antibody are administered to the patient to enhance an effective immune response (e.g., a HAXA response). In certain embodiments, preferably about 25 μg/kg to about 1 mg/kg of the non-specific xenotypic antibody are administered to the patient.

Administration of the non-specific xenotypic antibody that results in an enhanced effective immune response in the patient, which may be specific or non-specific, and allows the patient's immune response to be specifically stimulated against an antigen, preferably a disease-associated antigen.

In certain embodiments, the non-specific xenotypic antibody elicits an enhanced effective immune response in the patient wherein a patient's immune response is increased as compared to the immune response prior to the administration of the xenotypic antibody. Standard methods of detecting an increased immune response are known, such as an ELISA assay. For example, the antibody titer of a patient may be determined before and after the patient is administered the xenotypic antibody according to the methods of the invention, where an increased concentration of antibodies after administration as compared to before administration indicates an increased immune response. Similarly, a patient may exhibit an increased antigen-specific immune response (e.g., against a disease-associated antigen) regardless of whether the antigen-specific immune response arose following an increased non-specific or specific immune response and/or an increased HAXA response.

Antigen-specific immune responses are hallmarked by T cell responses, B cell responses, or preferably both against a particular antigen, such that the concentration of the antigen in the patient after administration of the compositions is reduced as compared to the concentration of the antigen prior to administration of the composition. Preferably, the reduction is at least 2-fold. Preferably, a patient who has developed an antigen-specific immune response comprises at least a T cell that specifically recognizes the antigen via a T cell receptor in the context of an MHC molecule, or an antibody produced by a B cell that specifically binds to the antigen.

In the present invention, a patient's T cell binds to and responds to an antigen, or fragment thereof, presented in context of a syngeneic MHC molecule. Thus, a T cell that specifically recognizes an antigen will bind to a fragment of the antigen in association with the MHC molecule. Subsequently, the T cell will respond by proliferating, secreting cytokines, and/or changing its cell surface phenotype (e.g., up-regulating CD69 expression). Thus, a patient's T cell that specifically recognizes the antigen will bind to and respond to the antigen when the antigen is in the context of an antigen presenting cell.

In certain embodiments of the invention, the patient generates a T cell that specifically recognizes the antigen. Identification of the T cell may be made by comparing T cells collected from the patient. In one embodiment, T cells (preferably CD8+ T cells) from the patient are collected and screened in a ⁵¹Cr-release assay (see Current Protocols in Immunology, supra). In certain embodiments of the invention, the patient generates a CD4+ T cell that specifically recognizes the antigen. Preferably, the CD4+ T cell is a helper T cell that specifically recognizes the antigen (or fragment thereof) in the context of a MHC molecule. In preferred embodiments, the patient generates both a CD4+ T cell that specifically recognizes the antigen and a CD8+ T cell that specifically recognizes the antigen.

In certain preferred embodiments of the methods according to the invention, CD4+ cytokine producing T cells (i.e., helper T cells) provide necessary immunological help (e.g., by stimulating release of cytokines) to induce and maintain not only CTL, but also a humoral immune response mediated by B cells. Thus, in certain embodiments of the methods according to the invention, a humoral response to the antigen is activated in the patient administered the composition.

Accordingly, if the patient received more than one dosage of the composition, the T cell that specifically recognizes the antigen may appear after any one or more of the numerous dosages of the composition administered to the patient. Likewise, if the patient received more than one dosage of the composition, an antibody that specifically binds to the antigen may appear after any one or more of the numerous dosages of the composition administered to the patient.

In certain embodiments of the invention, the patient generates a humoral (e.g., B cell) response. Identification of an antibody that specifically binds to the antigen after the administration of the composition is readily performed by standard immunological techniques. In one non-limiting example, sera from a patient prior to administration of the composition is collected and compared to sera from the patient after administration of the composition with regard to the presence or concentration of antibodies that specifically bind to the antigen. Such comparison may be made using, for example, an enzyme-linked immunosorbant assay (ELISA) with the antigen.

In certain embodiments, administration of xenotypic antibodies elicits a host anti-xenotypic antibody (HAXA) response in the patient. HAXA may result from an increased non-specific immune response in the patient administered the xenotypic antibody, or it may be from a specific immune response in the patient directed against the administered xenotypic antibody. It will be understood that the invention includes the generation of a HAXA response regardless of how the HAXA response is generated. For example, if the patient is a human and the xenotypic antibodies are rabbit antibodies, that HAXA is human anti-rabbit antibodies. Preferred HAXA include, without limitation, human anti-mouse antibody (HAMA).

In certain preferred embodiments, the level of HAXA rises to more than 100 times the level that was present before the administration of xenotypic antibody that does not specifically bind to the disease-associated antigen. In certain preferred embodiments, the level of HAXA rises to more than 10 times the level that was present before the administration of xenotypic antibody. In certain preferred embodiments, the level of HAXA rises to more than 3-fold the level that was present before the administration of xenotypic antibody within two weeks of administration of xenotypic antibody. In certain preferred embodiments, the level of HAXA rises to more than twice the level that was present before the administration of the xenotypic antibody after at least 3 injections of xenotypic antibodies. In certain preferred embodiments, the HAXA response is at least 200 ng antibody/ml serum. In certain preferred embodiments, the HAXA response is at least 5,000 ng antibody/ml serum.

In certain preferred embodiments, the composition administered to the patient further includes host anti-xenotypic antibodies (HAXA). In this embodiment, where the xenotypic antibody is administered with HAXA, the HAXA is preferably directed against the species of animal from which the xenotypic antibody is derived. Thus, if a murine antibody is administered to a human patient, the accompanying HAXA are human anti-murine antibodies (i.e., HAMA).

In certain embodiments, administration of the composition increases presentation of the antigen by an antigen-presenting cell in the patient by MHC class I or MHC class II. The antigen-presenting cell presents an antigen to CD4+ T cells (which are mostly helper T cells) and/or CD8+ T cells (which are mostly cytotoxic T cells). Thus, an antigen-presenting cell increases presentation of the antigen in context of class I and/or class II MHC after the patient has been administered an antibody as compared to the ability of the cell to present antigen before the patient was administered the antibody. Such an increased ability can be readily measured by available techniques (see Current Protocols in Immunology, ed. John E. Coligan, John Wiley & Sons, Inc. 2000). One exemplary technique to measure increased ability of an antigen-presenting cell to present antigen is an in vitro T cell activation assay, where ³H-thymidine uptake by patient T cells exposed to patient antigen-presenting cells is assessed.

C. Pharmaceutical Packages/Kits

One embodiment of the present invention is a pharmaceutical package, or kit, comprising a pharmaceutical composition comprising a non-specific antibody labeled for use as an adjuvant to enhance an immune response. A further embodiment, the package/kit includes instructions for use and administration. In a preferred embodiment of the present invention, the adjuvant of the pharmaceutical package/kit is a xenotypic antibody. In a further invention, the xenotypic antibody is a xenotypic monoclonal antibody. Specific examples of antibodies include, for example, Alt-1, Alt-2, Alt-3, Alt-4, Alt-5, and Alt-6.

The pharmaceutical package/kit of the instant invention may also contain a specific antibody immunoreactive with a disease-associated antigen labeled for use in treatment of a disease associated with an antigen to which the antibody binds. In a preferred embodiment of the present invention, the specific antibody of the pharmaceutical package/kit is a xenotypic antibody. In a further invention, the xenotypic antibody is a xenotypic monoclonal antibody. Specific examples of antibodies include, for example, Alt-1, Alt-2, Alt-3, Alt-4, Alt-5, and Alt-6.

The compositions of the pharmaceutical package/kit of the present invention can be formulated in single or multiple dose volumes such that the compositions can be administered to a patient as needed in order to enhance an immune response.

In preferred embodiments, the compositions of the pharmaceutical package/kit are approved for treatment of human patients and are free of pyrogens.

D. Vaccines

One embodiment of the present invention is a vaccine formulation to be administered to a patient having a disease or condition associated with an antigen to which an immunogenic response is desired. In one embodiment, a vaccine of the present invention includes a pharmaceutical composition of a disease associated antigen to which an immunogenic response is desired in combination with a non-specific xenotypic antibody which does not specifically bind to the disease associated antigen. Antigens and non-specific antibodies of the present invention have been described supra. In one exemplary example, a vaccine of the present invention includes a combination of a pharmaceutical composition of prostate specific antigen (PSA) and a pharmaceutical composition of Alt-1, which does not specifically bind to PSA. Administration of the vaccine induces an immune response to PSA that is non-specifically enhanced by Alt-1.

E. Administration

These materials may be administered orally; or by intravenous injection; or by injection directly into an affected tissue, as for example by injection into a tumor site, or intraperitoneally, intradermally, or subcutaneously; or by any suitable method known in the art.

Compositions of the present invention are preferably administered in a therapeutically effective amount such that an effective immune response as described above is elicited.

F. Exemplary Diseases for Treatment

Some non-limiting examples of such antigens associated with a disease include the prostate specific antigen (associated with prostate cancer), BRCA-1 and BRCA-2 antigens (associated with many adenocarcinomas, including breast cancer, lung cancer, and pancreatic cancer), CA125 (associated with ovarian cancer), aberrant myelin basic protein (associated with Alzheimer's disease or Multiple Sclerosis), gp120 (associated with colorectal, stomach, and pancreatic cancers), TAG-72 (associated with ovarian, stromal, and pancreatic cancers) and p53 (associated with various cancers).

### IV. EQUIVALENTS

Those skilled in the art will recognize, or be able to ascertain using no more than routine experimentation, many equivalents to the specific embodiments of the invention described herein. Such equivalents are intended to be encompassed by the following claims.

All of the above-cited references and publications are hereby incorporated by reference in their entireties.

### EXAMPLES

The following examples are intended to further illustrate certain particularly preferred embodiments of the invention and are not intended to limit the scope of the invention. The contents of all cited references (including literature references, issued patents, published patent applications as cited throughout this application) are hereby expressly incorporated by reference.

The practice of the present invention will employ, unless otherwise indicated, conventional techniques of cell biology, cell culture, molecular biology, microbiology and recombinant DNA, which are within the skill of the art. Such techniques are explained fully in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); and B. Perbal, A Practical Guide To Molecular Cloning (1984).

### Example I

### Effect Of HAMA On Antigen Uptake By Dendritic Cells

Complexation of antibody with specific antigen or with HAMA and binding to dendritic cells was measured. To do this, human anti-mouse antibody (HAMA) was purified from patient serum samples with high HAMA concentrations after MAb Alt-2 injection via affinity chromatography on Protein G, and a MAb-Alt-1 column (Alt-1 specifically binds to the MUC-1 antigen) to eliminate Ab2 (i.e., human antibody that binds to the idiotype of the MAb Alt-2 antibody).

Five micrograms (5 μg) of fluorescein (FITC)-labeled Alt-2 (anti-CA125) or Alt-6 (anti-PSA) murine monoclonal antibody was incubated together with the corresponding antigen (1 μg) and/or HAMA (2.5 μg) and 5×10⁵ immature dendritic cells at 37° C. for 60 minutes. The mixture was then washed once and resuspended in 0.5% formalin+PBS and subjected to flow cytometry (FACScan) on a FACSCalibur machine (Becton-Dickinson).

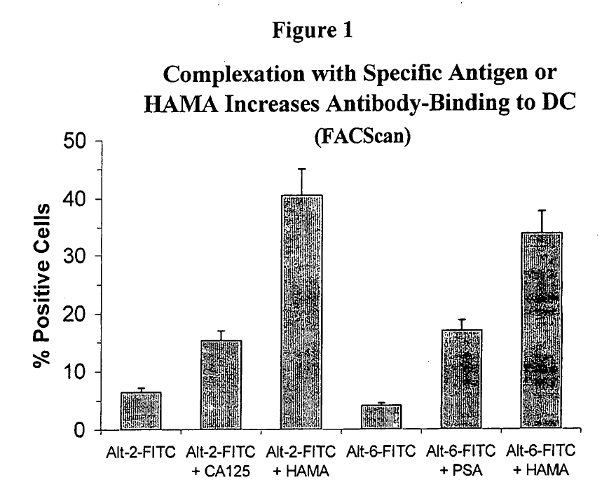

As shown in FIG. 1, antibody plus antigen bound to dendritic cells better than antibody alone; however, the highest binding was observed when the dendritic cells were exposed to antibody plus HAMA.

In another study, 0.3 to 2.5 μg/mL of FITC-labeled Alt-2 murine monoclonal antibody was incubated together with 8000 U/mL of unlabeled CA125 or 8000 U/mL of FlTC-labeled CA125 was incubated with 0.3 to 2.5 μg/mL of unlabeled Alt-2 with or without HAMA (0.33 to 2 μg/mL) and 5×10⁵ immature dendritic cells at 37° C. for 60 minutes. The binding to cells was assessed by flow cytometry.

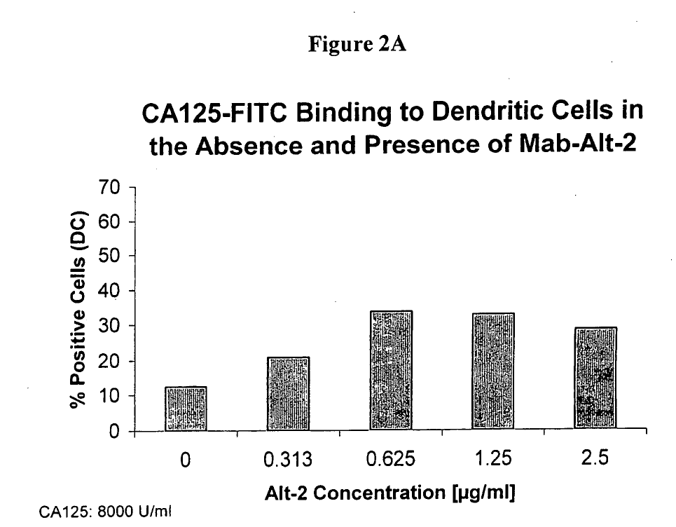

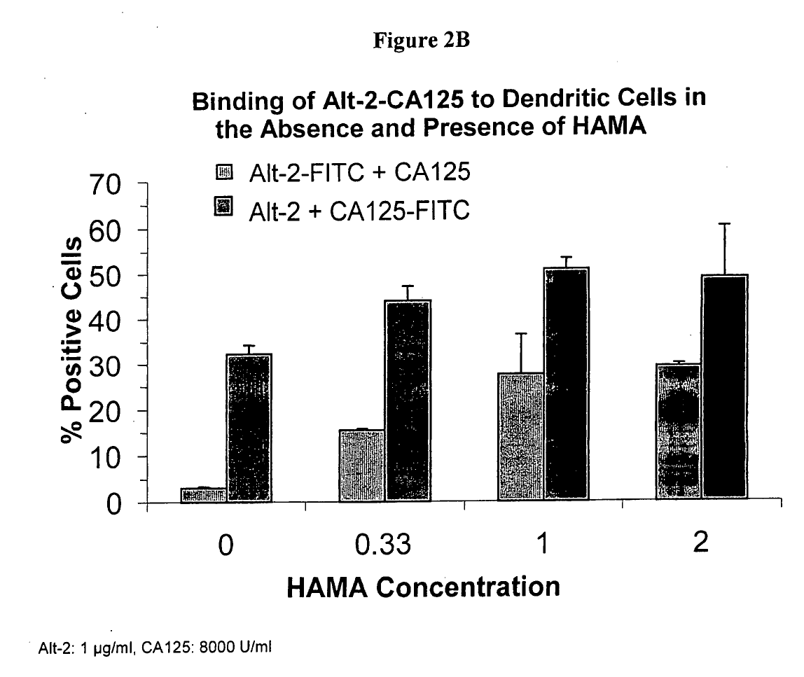

As shown in FIG. 2A, the addition of Alt-2 enhanced binding of FITC-labeled CA125 to dendritic cells. As shown in FIG. 2B, antigen/antibody immune complexes, regardless of whether the antigen (CA125) or the antibody (Alt-2) was FITC labeled, showed improved binding to dendritic cells in the presence of HAMA.

### Example II

### Effect Of HAMA On Antigen Uptake By Monocytes

In another experiment, CA125 or MAb-Alt-2 was labeled with FITC and incubated at 1000 U/ml of CA125 and 1 μg/ml of MAb-Alt-2 with monocytes for 1 hour at 37° C. The binding studies were conducted in the absence and presence of HAMA at three concentrations. The binding to cells was assessed by flow cytometry.

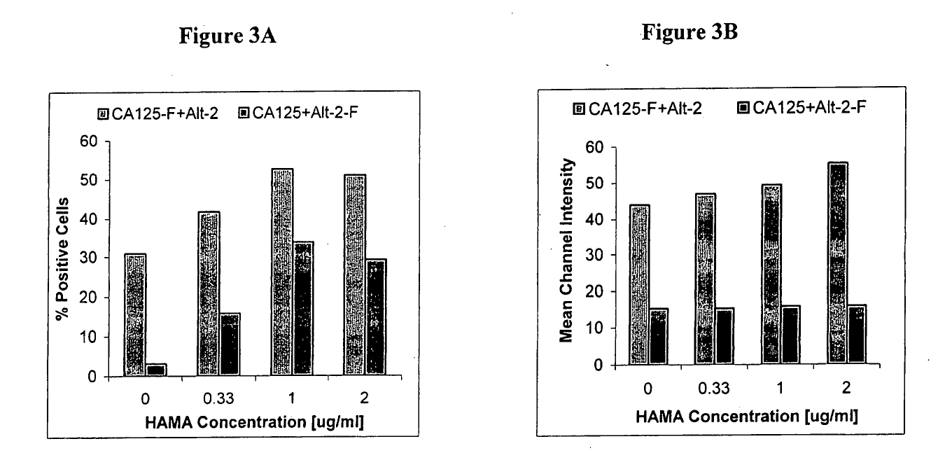

As shown in FIGS. 3A and 3B, both the CA125 antigen as well as the Alt-2 murine monoclonal antibody are taken up more efficiently in the presence of HAMA. FIG. 3A shows the increase in the number of monocytes bound by either FITC-labeled CA125 or FITC labeled Alt-2 in the presence of 0, 0.33, 1, or 2 μg/ml HAMA; FIG. 3B shows the increase in overall fluorescence per cell.

### Example III

### Effect of HAMA on Antigen Uptake By APCs

Further binding studies were conducted to compare the uptake of the CA125-MAb-Alt-2 complex by monocytes or immature dendritic cells in the presence and absence of equimolar amounts of HAMA. In this study, FITC-labeled CA125+ murine MAb-Alt-2 was incubated at 1000 U/ml of CA125 and a range of MAb-Alt-2 concentrations (0, 0.05, 0.1, 0.2, and 0.4 μg/ml) with monocytes or immature dendritic cells for 1 hour at 37° C. The binding studies were conducted in the absence and presence of human anti-mouse antibodies (HAMA). The HAMA concentrations were equivalent to the MAb-Alt-2 concentrations to form equimolar complexes of HAMA and MAb-Alt-2. Binding to cells was assessed by flow cytometry.

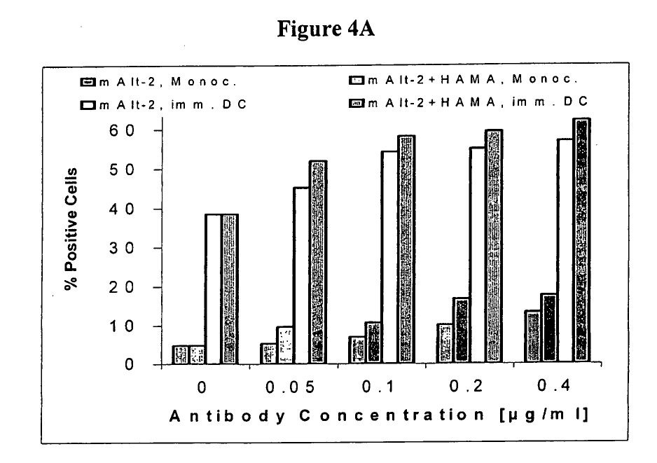

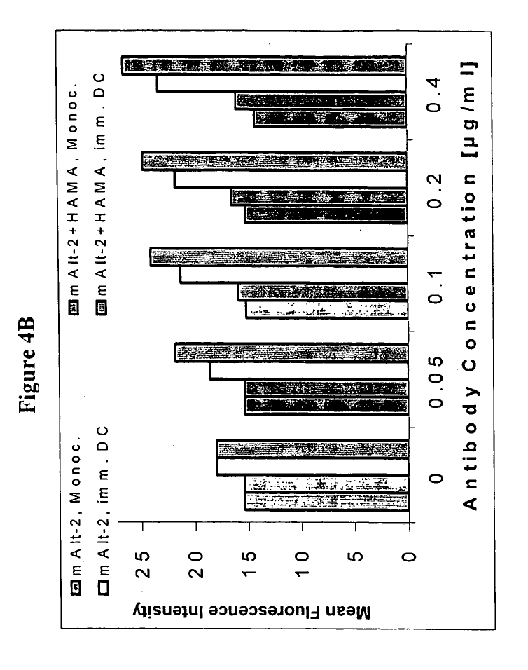

As shown in FIGS. 4A (percent positive cells) and 4B (mean fluorescence intensity), HAMA further enhanced the binding of CA125 to human monocytes and dendritic cells. Immune complexes with HAMA increased the percentage of CA125-targeted cells within the monocytes and dendritic cell populations (FIG. 4A), but increased the CA125 concentration per cell mainly in dendritic cells (FIG. 4B).

### Example IV

### Effect Of Xenotypic Antibody On Tumor Growth

Mice suffering from a solid, palpable tumor and having circulating free antigen from the tumor are administered varying amounts of a human antibody that specifically binds bovine serum albumin by intravenous injection. The mice are assayed at various time-points after administration for the development of mouse anti-human antibodies (MAHA). The mice are also assessed routinely for tumor size by caliper measurement and are compared to control mice with tumor and circulating antigen that are not administered the human antibody.

The mice are also assessed for survival from the tumor.

Eventually, all remaining living mice are euthanized and their final HAMA concentration and tumor volume are assessed.

### Example V

### Animal Model

SCID/bg mice (commercially available from Taconic, Germantown, N.Y.) are reconstituted with human PBL using the following method. Briefly, human peripheral blood leukocytes (PBLs) are isolated from human buffy coat or leukopheresis packs on a Ficoll-Hypaque gradient according to standard methods. Human PBLs are washed, resuspended in sterile phosphate buffered saline, and injected intraperitoneally into mice at a concentration of 1-3×10⁷ cells per mouse.

Two days later, all animals are implanted with a human tumor cell line that expresses the CA125 antigen (e.g., NIH OVCAR-3 cells commercially available from the American Type Culture Collection, Manassas, Va.) on day 0. The mice are next divided into three groups: Group 1 receives one injection of the non-specific control antibody MOPC-21 (commercially available from Sigma, St. Louis, Mo.) on day 4 (IP); Group II receives one injection of the non-specific control antibody MOPC-21 on day 4 (IP); and subsequently injections of the CA125-specific antibody Alt-2 on days 11, 18, and 25 (IP) and group III receives no injection of the non-specific control antibody MOPC-21 on day 4 but injections of the CA125-specific antibody Alt-2 on days 11, 18, and 25 (IP). The mice in all groups are followed for tumor growth and tumors are measured with every other day as soon as they appear. The mice in all groups are weighed every day as well. When the tumor becomes large and the overall appearance of the animal evidences discomfort, animal are sacrificed, the tumors excised and weighed, and the blood and spleen taken for T cell proliferation studies. T cell proliferation studies are performed against murine IgG and CA125 according to standard methods.

The mice of Group II (one injection of MOPC-21 (day 4) and injections of Alt-2 on days 11, 18, and 25) are found to have the smallest tumors. Mice in Group III are found to have slightly larger tumors than in Group II, and mice in Group I have the largest tumor of the three groups. In addition, the strongest HAMA and T cell proliferation response to mouse IgG and CA125 is detected in Group II, with the next-strongest response in Group III, and the weakest HAMA and T cell proliferation response is detected in Group I.

### Example VI

### Animal Model Using Xenotypic Non-specific and Specific Antibody to Inhibit Tumor Growth

SCID/bg mice were reconstituted with human peripheral blood lymphocytes (hPBL, normal donors) by intraperitoneal (IP) injection of 2-3×10⁷ hPBL/mouse. An isotype-matched control antibody without tumor-specificity (MOPC-21 or MAb-170) and PBS injection served as controls. The tumor-specific antibody Alt-2 and control antibody was administered at 100 μg/mouse in PBS, in different experimental designs.

Human-PBL-SCID/bg mice were either immunized before injection of tumor cells or along with tumor cells. Antibody injections were repeated one to three times in two-week intervals. Reconstitution with hPBL was confirmed both in the middle of the study and at termination by analysis of human serum immunoglobulin G (IgG) levels and demonstrated the successful engraftment of a human immune system. The ovarian cancer cell line NIH:OVCAR-NU-3 was injected IP at 1×10⁶ cells/mouse or subcutaneously (SC) at 2 to 4×10⁶ cells/mouse.

All four experiments showed that Alt-2 treatment (a) delayed or prevented development of tumors, (b) delayed tumor growth when injected prior to tumor implantation, and (c) prolonged the survival of the mice (IP tumor injection). To a lesser extend, the non-specific control antibody also had an effect on tumor growth. In study #1 (tumor size endpoint), female SCID/bg mice (9 mice per group) were reconstituted with humoral peripheral blood lymphocytes (hPBL) 7 days prior to tumor cell implantation. NIH:OVCAR-3 tumor cells were implanted subcutaneously on Day 0. Test antibodies or control agents were administered 7 days before and 7, 21 and 36 days following tumor cell implantation and included the following groups: 100 μg of Alt-2, 100 μg of control antibody MOPC-21 or PBS. Tumor measurements were taken throughout the course of the study. The effect of the treatment was determined by comparison of tumor volume in animals receiving treatment agents vs. animals receiving control agents.

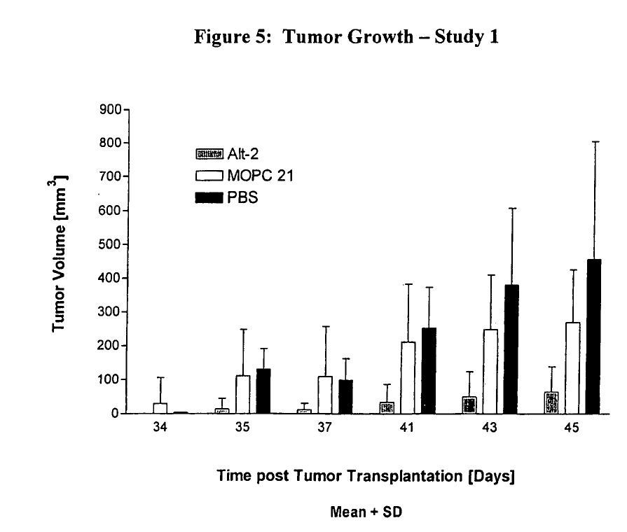

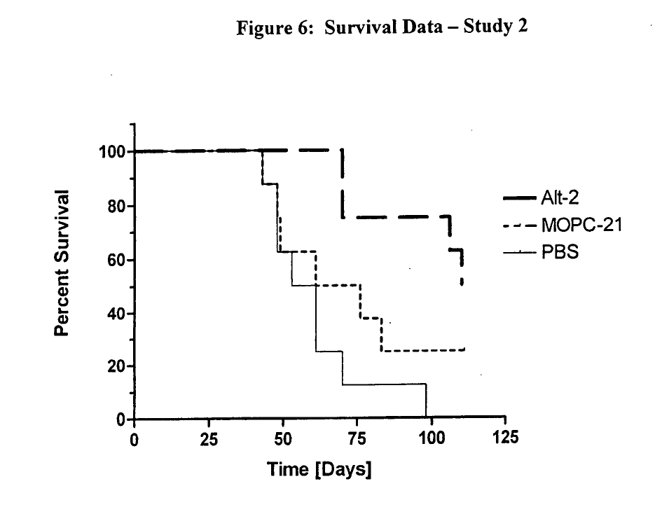

In Study 1, tumor volumes were significantly lower in mice receiving the specific antibody as compared to mice receiving PBS (p<0.05, Mann-Whitney U test, FIG. 5). Mice administered the non-specific control antibody also had smaller tumors (e.g., tumor volume and weight) compared to the mice receiving PBS; however, tumors were bigger compared to the mice receiving the specific antibody MAb-Alt-2.

In study 2 (survival endpoint), male SCID/bg mice were reconstituted with hPBL 21 days prior to tumor cell implantation. NIH:OVCAR-3 tumor cells were implanted intraperitoneally on Day 0. Test antibodies or control agents (Alt-2, MOPC-21 and PBS) were administered 21 and 14 days prior to tumor cell implantation and 5, 19 and 34 days post tumor cell implantation. Animals were observed every other day for signs of distress, discomfort and presence of intra-abdominal tumor. Sick animals were euthanized and dissected to determine the presence and extent of tumor mass. The effect of the treatment was determined by comparison of survival times in animals receiving treatment agents vs. animals receiving control agents.

Control animals treated with PBS had median survival of approximately 57 days. Mice treated with the non-specific control antibody MOPC 21 showed a median survival of 68.5 days. Animals treated with the specific Alt-2 antibody appeared to have the longest survival with a median of 111 days (FIG. 6).

In the third study, SCID/bg mice were implanted with NIH:OVCAR-3 tumor cells by subcutaneous (SC) injection. Four groups of mice (7 mice/group) were injected SC with 4×10⁶ NIH:OVCAR-NU-3 tumor cells into the right flank of the mice. Two weeks after tumor implantation (day 15), SCID/bg mice were reconstituted with 2×10⁷ hPBL/mouse and treated with 100 μg of Alt-2 or 100 μg of MOPC 21 control antibody by IP injections. Antibody injections were repeated once after two weeks (day 28). In mice with developing tumors, the tumor volumes (mm³) were determined by three-dimensional tumor measurements every other day with a caliper in the various groups.

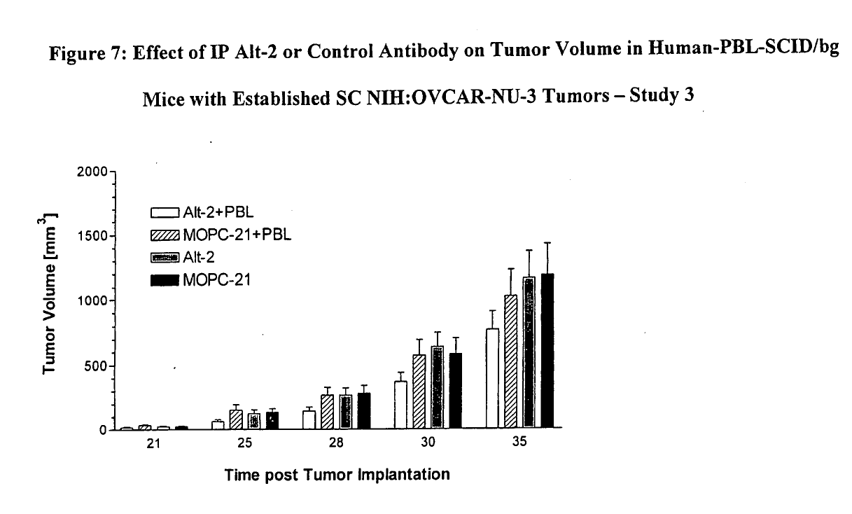

Treatment of tumor-bearing mice with IP Alt-2 (specific antibody) and hPBL significantly decreased the tumor growth compared to all control groups as evaluated by tumor size measurement (FIG. 7). Treatment with the non-specific control antibody MOPC-21 and hPBL also decreased the tumor growth compared to control groups without a human immune system (no huPBL).

In the fourth study, groups of SCID/bg mice (7 mice per group) were reconstituted IP with 2×10⁷ hPBL/mouse and treated with 100 μg of MAb-B43.13 or 100 μg of MAb-170 control antibody or PBS. The fourth group of mice (7 mice) without hPBL reconstitution received no further treatment and served as an additional control group referred to as “no treatment” group. One week after reconstitution and antibody treatment, all mice were implanted with 2×10⁶ NIH:OVCAR-3 tumor cells by SC injection. The mice in groups 1 to 3 received a booster with respective antibodies at Days 4, 26 and 45. In mice with developing tumors, the day of the appearance of the tumors and tumor volumes were recorded.

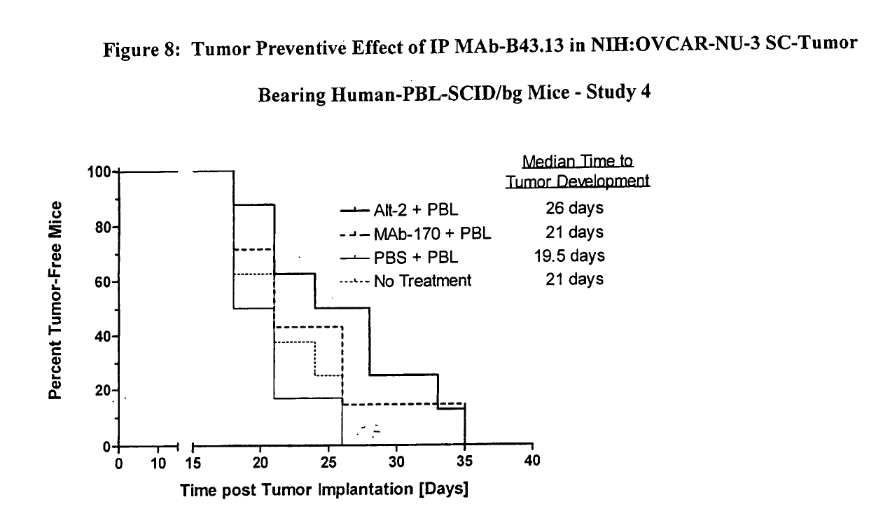

FIG. 8 shows the tumor appearance in mice injected with 2×10⁶ tumor cells for the various treatment groups. A consistent delay of tumor appearance in MAb-B43.13-PBL group was observed as compared to the control groups. Median tumor establishment was delayed by 5 days for MAb-B43.13 immunized mice over the isotype control group or the group with no treatment and by 6.5 days over the PBS group. Tumor establishment in 100% of the mice was delayed by nine days in MAb-B43.13-PBL or MAb-170-PBL groups compared to mice receiving PBS or no treatment. Treatment with the tumor-specific antibody showed the most delay in tumor appearance compared to the group with no treatment, however, mice treated with the MAb-170 non-specific control antibody (in the presence of a human immune system) also showed a delay of tumor appearance.

### Example VII

### Animal Model Using Xenotypic and Syngeneic Specific Antibody to Inhibit Tumor Growth

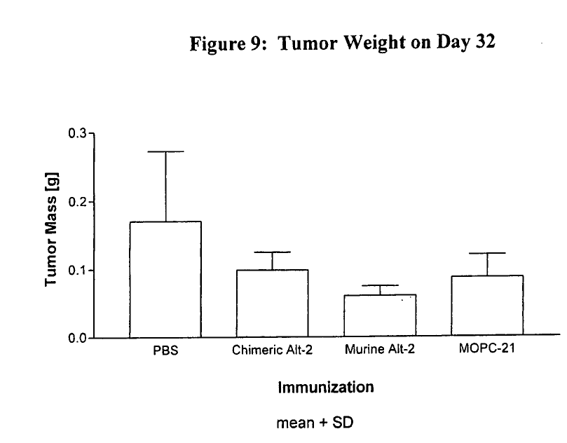

Female SCID/bg mice were reconstituted with hPBL 14 days prior to tumor cell implantation. NIH:OVCAR-3 tumor cells were implanted subcutaneously on Day 0. Test antibodies or control agents were administered 14 and 7 days prior to tumor cell implantation and following tumor cell implantation on days 7, 14 and 21. Four groups of 7 to 8 mice were injected intravenously with 50 μg/mouse/treatment of murine Alt-2, human-chimeric Alt-2, non-specific mouse IgG control antibody (MOPC-21) and PBS. Tumor measurements were taken during the course of the study and final tumor weights were determined at termination. Reconstitution with hPBL, confirmed at termination by analysis of human serum immunoglobulin G (IgG) levels, was successful. As shown in FIG. 9, final tumor weights were lowest in mice administered the treatment agent murine Alt-2. Mice administered chimeric Alt-2 or non-specific mouse antibody showed larger tumors than mice treated with murine Alt-2, however, final tumor weight were smaller than in mice receiving control PBS. Tumor growth was less in mice treated with the specific xenotypic antibody (murine Alt-2 in mice carrying a human immune system) as compared to the syngeneic antibody (human chimeric Alt-2) and was less in mice treated with non-specific xenotypic antibody (MOPC-21) as compared to the PBS control.

### Example VIII

### Animal Model Using Xenotypic and Syngeneic Specific as well as Non-specific Antibody to Inhibit Tumor Growth

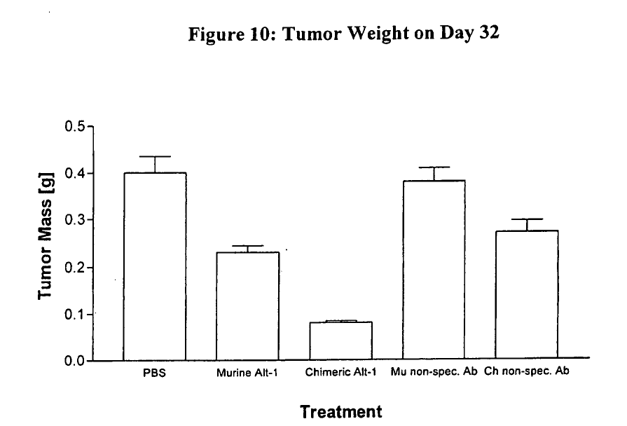

Female 6-week old BALB/c mice were grouped into 5 groups of 8 mice. The MUC-1 expressing murine tumor line 413BCR was implanted subcutaneously on Day 0 (2.5×10⁶ cells/mouse). Test antibodies or control agents were administered 14 and 7 days prior to tumor cell implantation and following tumor cell implantation on days 7, 14 and 21. Groups were injected subcutaneously with 50 μg/mouse/treatment of murine Alt-1, human-chimeric Alt-1, non-specific mouse IgG control antibody (MOPC-21), non-specific chimeric control antibody and PBS. The chimeric antibodies were generated by chemically conjugating human IgG to the murine antibodies Alt-1 and MOPC-21. Tumor measurements were taken during the course of the study and final tumor weights were determined at termination. As shown in FIG. 10, final tumor weights were lowest in mice administered the treatment agent chimeric Alt-1. Mice administered murine Alt-1 or non-specific chimeric antibody showed larger tumors than mice treated with chimeric Alt-1, however, final tumor weights were smaller than in mice receiving control PBS or control non-specific mouse IgG. Tumor growth was less in mice treated with the specific xenotypic antibody (human chimeric Alt-1 in a murine immune system) as compared to the syngeneic antibody (murine Alt-1) and was less in mice treated with non-specific xenotypic antibody (chimeric MOPC-21) as compared to the PBS control or the non-specific syngeneic antibody (MOPC-21).

### Example XI

### T Cell Responses To Dendritic Cells Loaded With Xenotypic Antibodies

Immature human dendritic cells (DC) are generated from monocytes of normal donors by culture in GM-CSF and IL-4. On day 4, immature DCs are loaded with medium control (Group 1 and 2) or MOPC-21 (5 μg/ml) (Group III) for 4 hours, and are subsequently matured by addition of TNF-alpha and IFN-alpha to the growth medium for three additional days. Autologous purified T cells are added on day 7 and incubated with the DC of Group II and Group III for another 7 days.

In the meantime, fresh immature DCs are prepared and loaded with Alt-2 (5 μg/ml) plus CA125 (500 U/ml) or medium on day 4 and matured as described above. The medium-loaded DCs are added to Group I (control group), then Alt-2 plus CA125 loaded DCs are added to the T cell cultures of Groups II and III on day 14 of the experiment and incubated for another 7 days.

In the mean time, another set of fresh immature DCs are prepared and loaded with Alt-2 (5 μg/ml), CA125 (500 U/ml), Alt-2 (5 μg/ml) plus CA125 (500 U/ml) or medium (negative control) on day 4 and matured as described above. Each group (Groups 1, 2, and 3) is split up into 4 subgroups and each set of the loaded DC is added to the four subgroups of 1, 2, and 3 on day 21 of the experiment and incubated for 2 hours. Next, the cells are blocked with Brefeldin A to stop secretion process (using the GolgiPlug™ reagent commercially available from BD PharMingen, San Diego, Calif., according to manufacturer's instructions), and incubated for additional 16 hours. At that point cells are harvested, transferred to FACS tubes, and stained for human CD3-FITC and human CD8-Cytochrome. Cells are washed, permeabilized with Saponin and reagents commercially available from BD PharMingen, and stained for intracellular IFN-γ with anti-human IFN-γ-PE. Cells are washed and fixed and then analyzed on a flow cytometry by gating on the CD3+ T cells.

Based on their expression of intracellular IFN-γ, the T cells of Groups 2 and 3 are found to respond to Alt-2, CA125 and the Alt-2-CA125 complex. Responses in Group 3 cells, which were originally exposed to control murine antibody MOPC-21 are found to be higher than in Group 2, which were originally exposed to medium only, after stimulation with CA125, Alt-2, and Alt-2-CA125 complexes. Responses in Group I are weak to non-detectable.

### Example X

### Human Studies

Case 1: A 63 year old female with stage III ovarian cancer is imaged with Oncoscint™ MAb 72.3 (commercially available from Cytogen Corp., Princeton, N.J.) as part of a diagnostic evaluation for vague abdominal symptoms during the period following complete remission of her disease achieved with carboplatinum and paclitaxel-based primary chemotherapy. The evaluation is negative, the symptoms are attributed to post surgical adhesions and the patient remains relapse-free for an additional 4 months. The patient develops a HAMA titre of 2000 ng/ml.

At the time of relapse the patient develops a rising level of CA125 antigen and is administered 2 mg of Alt-2 (available from AltaRex Corp., based in Edmonton, AB, Canada) three-times in 2-week intervals, and then quarterly, followed by platinum therapy (i.e., chemotherapy with a platinum-containing reagent) starting at week 6. The patient develops a vigorous immune response to CA125 and has a second complete clinical remission with the chemotherapy. The preexisting HAXA is attributed to augmenting the clinical effect of Alt-2 by enhancing the presentation of the complex.

Case 2: A 63 year old female with stage III ovarian cancer is imaged with Oncoscint™ MAb 72.3 (commercially available from Cytogen Corp., Princeton, N.J.) as part of a diagnostic evaluation for vague abdominal symptoms during the period following complete remission of her disease achieved with carboplatinum- and paclitaxel-based primary chemotherapy. The evaluation is negative, the symptoms are attributed to post-surgical adhesions and the patient remains relapse-free for an additional 4 months. In addition, as part of a clinical study, the patient receives AltaRex MAb Alt-1 as part of a BrevaRex PK study. The patient develops a HAMA titre of 2000 ng/ml in response to AltaRex MAb Alt-1.

At the time of relapse the patient develops a rising level of CA125 antigen and is administered 2 mg of Alt-2 three-times at 2-week intervals, and then quarterly, followed by Platinum therapy starting at week 6. The patient develops a vigorous immune response including boosting HAMA, Ab2, and T cells to CA125 and autologous tumor. The patient then enters a period of prolonged stable disease without further progression of her tumor.

1. A method for enhancing an immune response in a patient having a disease or condition, comprising administering to the patient a composition comprising a non-specific xenotypic antibody, wherein the xenotypic antibody does not specifically bind to an antigen associated with the disease or condition, whereby an immune response is enhanced.

2. The method of claim 1, further comprising administering to the patient a second composition comprising a specific xenotypic antibody that specifically binds to the antigen associated with the disease or condition.

3. The method of claim 2, wherein the non-specific xenotypic antibody enhances the immune response generated by the specific xenotypic antibody.

4. The method of any one of claims 1, 2, and 3, wherein administration of the non-specific xenotypic antibody composition elicits a host anti-xenotypic antibody (HAXA) response in the patient.

5. The method of claim 1, wherein the patient is a human.

6. The method of claim 1, wherein the non-specific xenotypic antibody is a murine antibody.

7. The method of claim 2, wherein the specific xenotypic antibody is a murine antibody.

8. The method of claim 6 or 7, wherein the specific murine antibody elicits a host anti-murine antibody (HAMA) response.

9. The method of claim 1, wherein administration of the non-specific xenotypic antibody increases presentation of an antigen associated with the disease or condition by an antigen-presenting cell.

10. The method of claim 3, wherein administration of the non-specific xenotypic antibody enhances an antigen-specific immune response in the patient.

11. The method of claim 10, wherein the antigen-specific immune response comprises generation of a T cell that specifically recognizes the antigen after administration of the composition.

12. The method of claim 11, wherein the T cell is a CD4+ T cell.

13. The method of claim 11, wherein the T cell is a CD8+ T cell.

14. The method of claim 1, wherein the non-specific xenotypic antibody composition further comprises a pharmaceutically acceptable carrier.

15. The method of claim 2, wherein the specific xenotypic antibody composition further comprises a pharmaceutically acceptable carrier.

16. The method of claim 1, wherein the non-specific xenotypic composition is administered in a dosage of from about 0.1 μg to about 2 mg of the xenotypic antibody per kilogram of body weight of the patient.

17. The method of claim 1 or 2, wherein the non-specific xenotypic antibody is a monoclonal antibody.

18. The method of claim 1 or 2, wherein the non-specific xenotypic antibody is a polyclonal antibody.

19. The method of claim 2, wherein the non-specific and the specific xenotypic antibodies are from the same species of animal.

20. The method of claim 2, wherein the non-specific xenotypic antibody composition is administered to the patient prior to the specific xenotypic antibody composition.

21. The method of claim 20, wherein the non-specific xenotypic antibody composition is administered to the patient one week prior to the specific xenotypic antibody composition.

22. The method of claim 20, wherein the non-specific xenotypic antibody composition is administered to the patient one month prior to the specific xenotypic antibody composition.

23. The method of claim 2, wherein the specific xenotypic antibody composition is administered to the patient prior to the non-specific xenotypic antibody composition.

24. The method of claim 23, wherein the specific xenotypic antibody composition is administered to the patient one week prior to the non-specific xenotypic antibody composition.

25. The method of claim 23, wherein the specific xenotypic antibody composition is administered to the patient one month prior to the non-specific xenotypic antibody composition.

26. The method of claim 2, wherein the non-specific xenotypic antibody composition and the specific xenotypic antibody composition are co-administered to the patient.

27. The method of claim 26, wherein the co-administration is in a single formulation.

28. The method of claim 26, wherein the co-administration is in two formulations.

29. The method of claim 10, wherein the specific antibody elicits an antigen-specific immune response comprising either a B cell with surface bound immnunoglobulin that specifically binds to the antigen after administration of the composition, or an antibody that specifically binds to the antigen after administration of the composition.

30. The method of claim 10, wherein the antigen-specific immune response comprises generating T cells that specifically recognize the antigen after administration of the composition.

31. The use of a non-specific xenotypic antibody as an adjuvant, wherein the non-specific antibody is not immunoreactive with an antigen for which an immunogenic response is desired.

32. The use of a non-specific xenotypic antibody in the formulation of a medicament for use as an adjuvant, wherein the non-specific antibody is not inununoreactive with an antigen for which an immunogenic response is desired.

33. A kit comprising the non-specific xenotypic antibody of claim 1 labeled for use as an adjuvant.

34. The kit of claim 33, further comprising the specific antibody of claim 2.

35. The method of claim 1, wherein enhancement of the immune response allows for an enhanced response against a disease-associated antigen.

36. A vaccine formulation including one or more antigens for which vaccination is desired and at least one xenotypic antibody not cross-reactive with the antigens of the vaccine, wherein the xenotypic antibody is provided in an amount to act as an adjuvant.
